(19) United States
(12) Patent Application Publication
(10) Pub. No.: US 2022/0395167 A1
(43) Pub. Date: Dec. 15, 2022

(54) FRONT-END STRUCTURE FOR INSERTION PART OF ENDOSCOPE

(71) Applicant: SCIVITA MEDICAL TECHNOLOGY CO., LTD., Suzhou, JS (CN)

(72) Inventor: Yi ZHANG, Suzhou (CN)

(73) Assignee: SCIVITA MEDICAL TECHNOLOGY CO., LTD., Suzhou, JS (CN)

(21) Appl. No.: 17/776,257

(22) PCT Filed: Nov. 13, 2019
(86) PCT No.: PCT/CN2019/117814

§ 371 Date: May 12, 2022

(51) Int. Cl. A61B 1/05; A61B 1/00; A61B 1/06

(52) U.S. Cl. CPC A61B 1/05; A61B 1/00096; A61B 1/0676

(57) ABSTRACT

The present invention relates to the field of medical endoscope, it discloses front-end structure for insertion part of endoscope, which consists of a probe shell a camera, a module arranged on the end surface of the probe shell to observe images of surrounding tissues; an operation hole arranged on the end surface of the probe shell and on the side of the camera module to fix the operation mechanism; the camera module consists of a lens unit and lighting units arranged on both sides of the lens unit, an arc transition surface is arranged on one side of the end surface of the probe shell to guide the probe to be inserted into the targeted tissue. The beneficial effects of the present invention are: a cut angle is arranged on the end surface of one end of the probe shell, when the probe is inserted into the human body, the inclined arc transition surface can reduce the resistance from the human body tissues, so that the probe can be inserted into the human body deeply in an easier manner; by arranging the probe shell and sealing the lighting units with sealant, the probe shell plays a role of positioning and protecting the ends of the lighting units.

### TECHNICAL FIELD

The present invention relates to the technical field of endoscope, particularly front-end structure for insertion part of endoscope.

### BACKGROUND TECHNOLOGY

As a medical device, medical endoscope is inserted into various cavities inside the human body and widely used in clinical observations and diagnoses as an import method of disease examination.

It is found through patent search that, a published patent under No. CN106308735A discloses an electronic cystoscope which consists of: a flexible tube, a rigid tube inside the flexible tube and a display, among which, a device channel, an endoscope channel and a lighting channel are arranged inside the rigid tube, a lens—an image sensor—is arranged inside the inner diameter channel, and a lamp is arranged inside the lighting channel.

However, in the above electronic cystoscope, no structure is arranged to position and protect the end parts of the lighting channel, during use, the secretions inside the human body may easily enter the endoscope probe, internal structural parts will be contaminated and damaged, shooting effects and service performances will be eventually affected; moreover, the outer side of the end panel of the above tracheoscope probe is a flat surface, when the probe and tube of the tracheoscope is inserted into the human body, the flat surface faces high resistance, and the tube is difficult to be inserted into the human body, so the probe may easily damage the tissues in the human body, in addition, when being inserted into the human body, the probe may easily be damaged due to high resistance.

### SUMMARY

The objective of the present invention is to design front-end structure for insertion part of endoscope to overcome the issue that both shooting effects and service performances of the medical endoscope in the prior art may easily be affected because the endoscope is contaminated and damaged when the secretions inside the human body enter the endoscope probe during use, as well as the problem that the tube is difficult to be inserted into the human body since the outer side of the end panel of the probe is a flat surface.

The technical scheme defined in the present invention to achieve above objective is, front-end structure for insertion part of endoscope, which consists of a probe shell, a camera module arranged on the end surface of the probe shell to observe images of surrounding tissues; an operation hole arranged on the end surface of the probe shell and on the side of the camera module to fix the operation mechanism; the camera module consists of a lens unit and lighting units arranged on both sides of the lens unit, an arc transition surface is arranged on one side of the end surface of the probe shell to guide the probe to be inserted into the targeted tissue.

As a reference embodiment, a camera fixing hole is arranged in the endoscope probe to assemble the camera module, the lens unit is arranged in the middle of the camera fixing hole, the lighting units are arranged on both sides of camera fixing hole, the lens unit and the lighting units are arranged in an triangle structure.

The specific inclination angle of the arc transition surface: The outer surface of the front end of the probe shell also consists of a lateral annular surface and an end surface, the included angle between the plane part of the arc transition surface and the end surface is 50°˜70°, preferably 60°.

The connection and fixing mode of the probe shell and the outer shell: Several claws are arranged on the end surface of the probe shell away from the camera module to buckle with the outer shell of the endoscope probe; the number of the claws is 4, the two claws close to the operation hole are the 1^{st} claws, and the claws close to the camera module are the 2^{nd} claws.

As an implementable scheme, mounting grooves corresponding to the camera module are arranged on the rear end surface of the probe shell, the lighting units are arranged inside the mounting grooves, the number of the mounting grooves is at least 2, and the mounting grooves are arranged on both sides of the lens unit.

As a preferred scheme, the part corresponding to the mounting grooves on the probe shell can be made of transparent material.

In order to guarantee the sealing of the lens, the probe shell and the camera module are connected and sealed with sealant, and the probe shell and the operation hole are connected and sealed with sealant.

The beneficial effects are,

The probe provided by the present invention, which consists of the probe shell, a cut angle is arranged on the end surface of one end of the probe shell, the end surface on which the cut angle is arranged forms an arc transition surface, and the arc transition surface is used to guide the probe to be inserted into the targeted tissue; when the probe is inserted into the human body, the inclined arc transition surface can reduce the resistance from the human body tissues, so that the probe can be inserted into the human body deeply in an easier manner, and the damages caused by the probe to tissues in the human body can be reduced; in addition, damages to the probe due to high resistance can be avoided.

The probe provided by the present invention, the operation hole is arranged on the probe shell, a surgery instrument mechanism is arranged inside the operation hole for surgical operation, or normal saline is introduced for flushing tissues and clearing vision field of the camera, more functions are achieved for the device.

Front-end structure for insertion part of endoscope provided by the present invention, by arranging the probe shell and sealing the lighting units with sealant, the probe shell plays a role of positioning and protecting the ends of the lighting units, during use of the endoscope, the secretions inside the human body contaminating the lighting units can be effectively prevented, the damages to the lighting units and the decline of detection effects can be avoided, high-quality shooting effects and practical performances can be achieved.

### DESCRIPTION OF THE DRAWINGS

FIG. 1 is an overall structural view of the present invention;

FIG. 2 is a structural view of the present invention without the camera module;

FIG. 3 is a sectional view of the structure of the present invention;

FIG. 4 is a rear view of the structure of the present invention;

In figures, 1. Probe shell; 2. Camera module; 201. Lens unit; 202. Lighting unit; 3. Camera fixing hole; 4. Arc transition surface; 5. Operation hole; 6. 1^{st} claw; 7. 2^{nd} claw.

### DESCRIPTION OF EMBODIMENTS

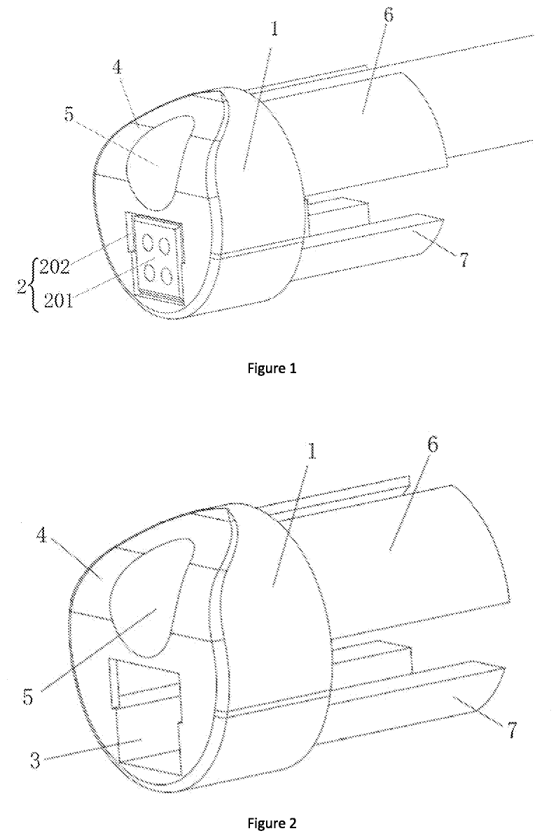

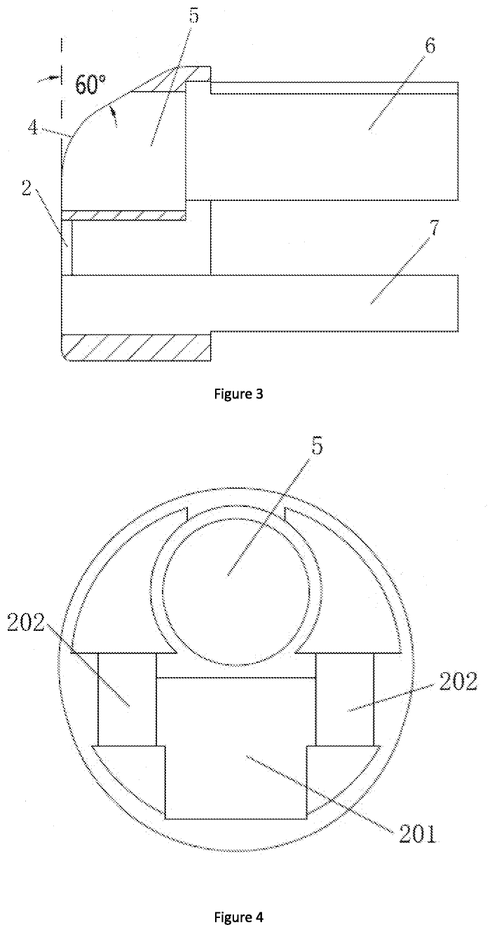

The present invention is described in detail below by combing attached drawings, as shown in FIGS. 1-4, an overall structural view of the present invention, which mainly consists of a probe shell 1, a camera module 2 and an operation hole 5, among which, the camera module 2 is the core mechanism of the endoscope and is arranged on the end surface of the probe shell 1 to observe images of surrounding tissues, the images are transmitted to the external display device for displaying images of surrounding tissues; the operation hole 5 is arranged to further expand the functions of the endoscope, it is arranged on the end surface of the probe shell 1 and on the side of the camera module 2 to fix the operation mechanism; the operation mechanism consists of the micro surgical instruments that may be involved in medical observation, or the liquid medicine used to treat the tissues, or the normal saline used to flush the mucus out of the tissues in order to assure clear images shot by the camera lens.

### Embodiment 1

An embodiment of the camera module 2 is described below, the camera module 2 consists of a lens unit 201 and lighting units 202 arranged on both sides of the lens unit 201; the lens unit 201 consists of a lens and a CMOS module connected with the lens, based on actual situation, the CMOS module is only an optional embodiment, other image transmission modules can be used, such as CCD module; about the lighting units 202, the lighting units 202 are mainly LED lamps, similarly, other cold light sources can be adopted for the lighting units 202. In order to fix the lens unit 201 and the lighting units 202 onto the probe shell 1, a camera fixing hole 3 is arranged in the endoscope probe to assemble the camera module 2; in order to ensure the sealing, the lens unit 201 and the probe shell 1 are sealed with sealant, the secretions inside the human body entering the device, affecting the use of the device and causing direct damages to the device under serious circumstance can be prevented during use. In order to achieve better shooting effects of the lens, this technical scheme provides a layout mode in which the lens unit 201 and the lighting units 202 are arranged inside the camera fixing hole 3, particularly, mounting grooves corresponding to the camera module 2 are arranged on the rear end surface of the probe shell 1, the lighting units 202 are arranged inside the mounting grooves, the number of the mounting grooves is 2, and the mounting grooves are arranged on both sides of the lens unit 201; the lens unit 201 is arranged in the middle of the camera fixing hole 3, the lighting units 202 are arranged on both sides of camera fixing hole 3, the lens unit 201 and the lighting units 202 are arranged in an triangle structure, the design of such structure can ensure sufficient lighting for the lens, avoid polarized lights and guarantee the quality of shot images.

In this embodiment, the diameter of the outer tube is D, and D meets 2.9 mm≤D≤3.1 mm; the ultra-small lens available on the market is used in the present invention, the size of the ultra-small lens meets the size requirements of the camera fixing hole 3, that is, it can be assembled inside the tube of above endoscope probe structure. Since the camera module 2 is arranged on the end surface of the endoscope, the endoscope probe structure defined in the present invention prevents arranging the camera module 2 on the operation end and collecting and transmitting images through optical fiber, greatly decreases the manufacturing cost of the endoscope since transmission through optical fiber is not required, can be sued as a disposal product, and eliminate the risk of surgical cross infections due to incomplete disinfection.

Another innovation point of the present invention is that, as shown in FIGS. 1˜3, an arc transition surface 4 is arranged on one side of the end surface of the probe shell 1 to guide the probe to be inserted into the targeted tissue. In addition to the arc transition surface 4, the external surface at the front end of the probe shell 1 also consists of a lateral annular surface and an end surface, the arc transition surface 4 is the transition surface between the lateral annular surface and the end surface, the transition surface inclines, particularly, the included angle between the plane part of the arc transition surface 4 and the end surface is preferably 60°; by arranging the arc transition surface 4 on the front end surface of the probe shell 1, the resistance facing by the endoscope probe when being inserted into the human body can be decreased, and damages to the tissues in the human body can be reduced.

In addition to the above mechanism, claws are arranged at the tail end of the probe shell 1 to fix the probe shell 1 inside the outer shell (the outer shell is the outmost shell of the endoscope, it is used to wrap the entire endoscope, and it is not shown in figures), the number of the claws is 4, the two claws close to the operation hole 5 are the 1^{st} claws 6, the claws close to the camera module 2 are the 2^{nd} claws 7, and the 1^{st} claws 6 and the 2^{nd} claws 7 have similar structures.

### Embodiment 2

This embodiment provides a probe, compared with the structure of the probe provided in embodiment 1, the only difference is that, the number of mounting grooves can be 1, 3, 4 and etc., accordingly, the number of the lighting units 202 arranged inside the mounting grooves can be 1, 3, 4 and etc., specific number is determined on the basis of the lighting intensity required for the endoscope.

### Embodiment 3

This embodiment provides a probe, compared with the structure of the probe provided in embodiment 1, the only difference is that, the entire probe shell 1 can be made of non-transparent material, only the end of the probe shell 1 is made of transparent material, the light emitted by the LED module passes through the end of the shell and illuminates the target tissues; as a further replaceable embodiment, the ordinary LED lamp is replaced by a waterproof LED lamp, a hole is arranged for the LED lamp at the end of the shell to expose the LED lamp, so that the light emitted by the LED module can directly illuminate the target tissues.

### Embodiment 4

This embodiment provides a probe, compared with the structure of the probe provided in embodiment 1, the only difference is that, the shape of the operation hole 5 can be diamond, circle and etc.; further, the operation hole 5 can extend outside onto the arc transition surface 4.

### Embodiment 5

This embodiment provides a probe, compared with the structure of the probe provided in embodiment 1, the only difference is that, the end of the operation hole 5 completely falls onto the arc transition surface 4.

It is obvious that the above embodiments are only examples for clear description but not a limitation of the embodiments. Changes or modifications in other forms can be made on the basis of the above description by those skilled in the art. It is impossible to describe all the embodiments. The obvious changes or modifications made on the basis of the above description fall in the protection scope of the present invention.

1. Front-end structure for insertion part of endoscope consisting of a probe shell, which is featured by:
A camera module, which is arranged on the end surface of the probe shell to observe images of surrounding tissues;
An operation hole, which is arranged on the end surface of the probe shell and on the side of the camera module to fix the operation mechanism;
The camera module consists of a lens unit and lighting units arranged on both sides of the lens unit, an arc transition surface is arranged on one side of the end surface of the probe shell to guide the probe to be inserted into the targeted tissue.

2. Front-end structure for insertion part of endoscope as recited in claim 1, which is featured by: a camera fixing hole is arranged in the endoscope probe to assemble the camera module, the lens unit is arranged in the middle of the camera fixing hole, the lighting units are arranged on both sides of camera fixing hole, the lens unit and the lighting units are arranged in an triangle structure.

3. Front-end structure for insertion part of endoscope as recited in claim 1, which is featured by: the outer surface of the front end of the probe shell also consists of a lateral annular surface and an end surface, the included angle between the plane part of the arc transition surface and the end surface is 50°˜70°.

4. Front-end structure for insertion part of endoscope as recited in claim 2, which is featured by: the included angle between the plane part of the arc transition surface and the end surface is preferably 60°.

5. Front-end structure for insertion part of endoscope as recited in claim 1, which is featured by: several claws are arranged on the end surface of the probe shell away from the camera module to buckle with the outer shell of the endoscope probe.

6. Front-end structure for insertion part of endoscope as recited in claim 5, which is featured by: the number of the claws is 4, the two claws close to the operation hole are the 1^{st} claws, and the claws close to the camera module are the 2^{nd} claws.

7. Front-end structure for insertion part of endoscope as recited in claim 1, which is featured by: mounting grooves corresponding to the camera module are arranged on the rear end surface of the probe shell, the lighting units are arranged inside the mounting grooves, the number of the mounting grooves is at least 2, and the mounting grooves are arranged on both sides of the lens unit.

8. Front-end structure for insertion part of endoscope as recited in claim 1, which is featured by: the section corresponding to the mounting grooves on the probe shell can be made of transparent material.

9. Front-end structure for insertion part of endoscope as recited in claim 1, which is featured by: the probe shell and the camera module are connected and sealed with sealant, and the probe shell and the operation hole are connected and sealed with sealant.
